(19) United States
(12) Patent Application Publication
(10) Pub. No.: US 2007/0190627 A1
(43) Pub. Date: Aug. 16, 2007

(54) Processes for making ethanol

(75) Inventors: Lori Henderson, Wake Forest, NC (US); Don Higgins, Franklinton, NC (US)

Correspondence Address: NOVOZYMES NORTH AMERICA, INC., 500 FIFTH AVENUE, SUITE 1600, NEW YORK, NY, 10110, US

(73) Assignee: Novozymes North America, Inc., Franklinton, NC (US)

(21) Appl. No.: 10/572,864

(22) PCT Filed: Sep. 15, 2004
(86) PCT No.: PCT/US04/30277

§ 371 Date: Mar. 1, 2007

Related U.S. Application Data
(60) Provisional application No. 60/504,034, filed on Sep. 19, 2003.

(51) Int. Cl. C12P 1/02; C12P 7/06; C12P 19/02

(52) U.S. Cl. 435/161; 435/171; 435/105

(57) ABSTRACT

An ergonomic chair system constructed from a stable base holding a rocking balanced seating structure, provided with an adjustable suspended relaxing supple seat, allowing an independent balanced controlled and secured inclination by body weight only without help, effort or locking device. This chair system is especially intended for the use of ergonomic chairs and particularly well adapted to users with reduced mobility thanks to its ergonomic seat, its autonomous use and the possible secured accompanying in the chair from and to the standing up position for users. This chair system is realizable in all usual materials of furnishing.

### FIELD OF THE INVENTION

The present invention relates to processes for making ethanol and other starch-based products.

### BACKGROUND OF THE INVENTION

The production of ethanol for fuel, beverages and industrial use is a major industry. Ethanol has widespread application, including for use as a gasoline additive or as a straight liquid fuel.

Ethanol manufactures employ two main processes in the production of ethanol and other starch-based products: wet milling and dry milling.

Wet milling and dry milling are very different processes and result in very different products and co-products. In wet milling, whole cereal grains are first steeped to loosen the outer fiber and then the grains are separated into the different fractions (starch, germ, fiber, oil and protein). In addition to ethanol, the wet milling process is used to produce a variety of diverse co-products, such as, starch, corn sweeteners, corn oil, high and low protein products and fiber.

In dry milling, whole cereal grains are ground in a substantially dry state, that is, without steeping the kernels to separate the kernels into their major components. The ground meal is liquefied, saccharified, fermented and optionally distilled to make ethanol. In addition to ethanol, the other major co-products of dry milling are the spent grains (termed distillers' grains) and carbon dioxide. Distillers' grains are produced from the de-alcoholized fermentation residues which remain after cereal grains have been fermented by yeast. Distillers' grains may be used as animal feed, but otherwise have a relatively low commercial value compared to the co-products produced in wet milling ethanol production.

Despite the low value of the distillers' grains co-product, dry milling is preferred over wet milling for ethanol production because a dry mill ethanol unit is much less capital and energy intensive than a wet mill ethanol unit. However, because of the relatively low value of the distillers' grains co-product, when raw material prices increase, the net raw material costs associated with dry-milling may become cost prohibitive.

It is therefore desired to improve the efficiency of especially dry milling ethanol production processes.

### SUMMARY OF THE INVENTION

The present invention provides improved processes for recovering components of distillers' grains for use in various applications. The present invention also provides processes for producing ethanol comprising in a dry milling step by using the components of distillers' grains, such as, the components of distillers' dried grains (DDG), distillers' dried grains with solubles (DDGS) and distillers' wet grains (DWG).

In one aspect the present invention provides processes for recovering components of distillers' grains, preferably, from distillers' dried grains (DDG). In a preferred embodiment of this aspect of the present invention, distillers' grains are treated with a fatty acid oxidizing enzyme. This is done to release the starch and/or non-starch components, in particular protein, present in distillers' grains. In a more preferred embodiment of this aspect of the present invention, distillers' grains are also subjected to a chemical treatment and/or mechanical treatment to promote the release of starch and/or non-starch components, in particular protein, from distillers' grains, preferably prior to or simultaneously with the fatty acid oxidizing enzyme treatment of the distillers' grains. The recovered starch and non-starch components may be separated and/or further processed for use, e.g., in ethanol production or as a nutritional supplement.

In another aspect the present invention provides processes for producing ethanol in which starch and/or non-starch components, in particular protein, present in distillers' grains, preferably, distillers' dried grains (DDG), are recovered from the distillers' grains and used for ethanol production. In a more preferred embodiment of this aspect of the present invention, distillers' grains are also subjected to a chemical treatment and/or mechanical treatment to promote the release of the starch and non-starch components, in particular protein, from distillers' grains, preferably prior to or simultaneously with the fatty acid oxidizing enzyme treatment of distillers' grains.

By utilizing residual starch present in distillers' grains, further improvements in ethanol yields can be obtained, and in particular, starch which was previously unavailable for use in ethanol production can now be utilized. The released starch is preferably treated with a starch degrading enzyme, preferably, a raw starch degrading enzyme, so as to convert the starch recovered from distillers' grains to oligosaccharides. The released starch and/or the treated starch are preferably fed into the ethanol process at a suitable location for further ethanol production, such as, at the liquefaction, saccharification and/or fermentation steps.

Although not limited to any theory of operation, improvements in ethanol production processes are obtained by recovering protein present in distillers' grains, treating the recovered protein with a protease and using the protease treated protein to improve fermentation efficiency by providing the yeast used for fermentation with improved nutritional benefits so as to thereby reduce the fermentation time. In a more preferred embodiment of this aspect of the present invention, distillers' grains are also subjected to a chemical treatment and/or mechanical treatment to promote the release of the protein, preferably prior to or simultaneously with the fatty acid oxidizing enzyme treatment. The released protein is preferably treated with a protease, so as to convert the protein to oligopeptides and amino acids. The released protein and/or the protease-treated protein may then be fed into the ethanol process stream at a suitable location, such as, at the liquefaction, saccharification and/or fermentation steps, more preferably directly to the fermentation step. In an embodiment of the invention the starch and protein released from distillers' grains are treated with a combination of a starch degrading enzyme, such as, a raw starch degrading enzyme, and a protease. The treated starch and protein may then be fed into the ethanol production stream at a suitable location, preferably during the saccharification and/or fermentation processes. The fatty acid oxiziding enzyme treatment of the invention may be combined with treatment with other enzyme activities such as esterases, preferably lipolytic enzymes, such as especially lipases or phospholipases, hemicellulases or cellulases.

### DETAILED DESCRIPTION OF THE INVENTION

Dry milling processes are well-known in the art, and generally involve the step of grinding/milling whole cereal grains in a dry or substantially dry state. The production of ethanol in accordance with a dry milling process generally includes the main process steps of grinding/milling whole cereal grains to produce a meal, and subjecting the meal to liquefaction, saccharification, fermentation, and optionally distillation to produce ethanol. Whole corn grains are the preferred starting raw material for ethanol production; however, other cereal grains may also be used, including, for example, milo, wheat and barley.

Liquefaction is a process in which the long chained starch is degraded into oligosaccharides. Liquefaction processes are well-known in the art, and are usually performed by enzymatic or acid hydrolysis. Preferably, liquefaction is preformed by treating the meal with an effective amount of an alpha-amylase. Liquefaction is often carried out at a temperature of about 105 to 110° C. for about 5 to 10 minutes followed by a lower temperature holding period of about 1 to 2 hours at 95° C.

Saccharification is a process in which the oligosaccharides resulting from liquefaction are converted by hydrolysis to monosaccharide sugars, such as dextrose. The hydrolysis is preferably preformed enzymatically by addition of a glucoamylase, alone or in combination with other enzymes, such as alpha-glucosidase and/or acid alpha-amylase. Saccharification processes are also well-known in the art. A full saccharification process may last about 72 hours, and is often carried out at temperatures from about 30 to 65° C. However, it is often more preferred to do a pre-saccharification step, lasting for about 40 to 90 minutes, and then to do a complete saccharification process during fermentation in simultaneous saccharification and fermentation (SSF) or simultaneous liquefaction, saccharification, and fermentation (LSF).

In the fermentation step, yeast is added to the mash to ferment sugars to ethanol and carbon dioxide. Preferred yeast includes strains of the genus Saccharomyces, more preferably, strains of Saccharomyces cerevisiae. Commercially available yeast include, e.g., Red Star®/Lesaffre Ethanol Red (available from Red Star/Lesaffre, USA), SUPERSTART (available from Alltech), GERT STRAND (available from Gert Strand AB, Sweden) and FERMIOL (available from DSM Specialties).

Fermentation processes are well-known in the art. Fermentation is generally carried out for about 24 to 96 hours, such as typically for about 35 to 60 hours. In preferred embodiments, the temperature is generally between 26 and 34° C., in particular about 32° C., and the pH is generally from pH 3 to 6, preferably around pH 4 to 5. Yeast cells are preferably applied in amounts of 10⁵ to 10¹², preferably from 10⁷ to 10¹⁰, especially 5×10⁷ viable yeast count per mL of fermentation broth. During the ethanol producing phase the yeast cell count should preferably be in the range from 10⁷ to 10¹⁰, especially around 2×10⁸. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference. In a continuous fermentation process, the fermenting mash will be allowed to flow, or cascade, through several fermentors until the mash is fully fermented and then leaves the final tank. In a batch fermentation process, the mash stays in one fermentor for an effective amount of time, for example, for about 48 hours, before distillation is started.

Distillation is a process of separating ethanol from the fermented mash, preferably, by evaporation. The vapors are preferably driven off by applying direct heat to the fermented mash. The vapors are collected, condensed and recovered as a liquid and may be redistilled to increase the ethanol concentration. Because ethanol has a higher vapor pressure than water, the vaporization of water and ethanol results in a liquid higher in ethanol. Through condensation, a highly concentrate distillate is obtained. Normal distillation results in a liquid with a purity of about 95 volume-% ethanol (190 proof). For fuel ethanol, the final proof must approach 200. To accomplish this result, the ethanol may be subjected to further dehydration steps.

Stillage is a product which remains after mash has been converted to sugar, fermented and distilled into ethanol. Stillage can be separated into two fractions, such as, by centrifugation or screening: (1) wet grain (solid phase) and (2) the thin stillage (supernatant). The solid fraction or distillers' wet grains (DWG) can be pressed to remove excess moisture and then dried to produce distillers' dried grains (DDG). After ethanol has been removed from the liquid fraction, the remaining liquid can be evaporated to concentrate the soluble material into condensed distillers' solubles (DS) or dried and ground to create distillers' dried solubles (DDS). DDS is often mixed with DDG to form distillers' dried grains with solubles (DDGS). DDG, DDGS, and DWG are collectively referred to as distillers' grains.

As shown in the table below, distillers' grains contain residual amounts of starch and other non-starch components, including protein. The residual starch and non-starch components are generally not accessible for use in ethanol production, as they are bound in the form of distillers' grains.

TABLE
Distillers' Grains Composition

| Components | Average % |
|---|---|
| Carbohydrate | |
| Non-starch glucan | 11.5 |
| Starch | 6.2 |
| Xylan | 10.9 |
| Galactan | 2.3 |
| Arabinan | 7.6 |
| Mannan | 1.5 |
| Total CHO | 40.1 |
| Other Components | |
| Protein | 32.9 |
| Acetyl groups | 1.6 |
| Ash | 2.6 |

In accordance with the present invention, the starch and/or non-starch components of distillers' grains, in particular protein, may be used for ethanol production. Preferably, starch and non-starch components of distillers' grains are recovered by enzymatic treatment of distillers' grains and then used for ethanol production, as described herein. More preferably, the starch and/or non-starch components of distillers' grains are recovered by a combination of chemical and/or mechanical treatment and enzymatic treatment processes, and then used for ethanol production, as described herein.

Enzymatic Treatment

In a preferred embodiment of the present invention, distillers' grains, preferably, distillers' dried grains (DDG), are treated with a fatty acid oxidizing enzyme in amounts effective to release residual starch and/or non-starch components, in particular protein, present in distillers' grains. Preferably, starch obtained from distillers' grains is treated with a starch degrading enzyme to convert the starch to oligosaccharides and other forms suitable for ethanol production. Treatment of the starch with starch degrading enzyme(s) may be carried out simultaneously with or subsequent to the fatty acid oxidizing enzyme treatment.

Fatty Acid Oxidizing Enzyme

The term “a” fatty acid oxidizing enzyme means at least one of such enzymes. The term “at least one” means one, two, three, four, five, six or even more of such enzymes.

In the present context, a “fatty acid oxidizing enzyme” is an enzyme which hydrolyzes the substrate linoleic acid more efficiently than the substrate syringaldazine. “More efficiently” means with a higher reaction rate. This can be tested using the method described in Example 2, and calculating the difference between (1) absorbancy increase per minute on the substrate linoleic acid (absorbancy at 234 nm), and (2) absorbancy increase per minute on the substrate syringaldazine (absorbancy at 530 nm), i.e., by calculating the Reaction Rate Difference (RRD)=(d(A₂₃₄)/dt−d(A₅₃₀)/dt). If the RRD is above zero, the enzyme in question qualifies as a fatty acid oxidizing enzyme as defined herein. If the RRD is zero, or below zero the enzyme in question is not a fatty acid oxidizing enzyme.

In particular embodiments, the RRD is at least 0.05, 0.10, 0.15, 0.20, or at least 0.25 absorbancy units/minute.

In a particular embodiment of the method of Example 2, the enzymes are well-defined. Still further, for the method of Example 2 the enzyme dosage is adjusted so as to obtain a maximum absorbancy increase per minute at 234 nm, or at 530 nm. In particular embodiments, the maximum absorbancy increase is within the range of 0.05-0.50; 0.07-0.4; 0.08-0.3; 0.09-0.2; or 0.10-0.25 absorbancy units per minute. The enzyme dosage may for example be in the range of 0.01-20; 0.05-15; or 0.10-10 mg enzyme protein per mL.

In the alternative, a “fatty acid oxidizing enzyme” may be defined as an enzyme capable of oxidizing unsaturated fatty acids more efficiently than syringaldazine. The activity of the enzyme could be compared in a standard oximeter setup as described in Example 1 below at pH 6 and 30° C. including either syringaldazine or linoleic acid as substrates.

In a particular embodiment, the fatty acid oxidizing enzyme is defined as an enzyme classified as EC 1.11.1.3, or as EC 1.13.11.-. EC 1.13.11.—means any of the sub-classes thereof, presently forty-nine: EC 1.13.11.1-EC 1.13.11.49. EC 1.11.1.3 is designated fatty acid peroxidase, and EC 1.13.11.—is designated oxygenases acting on single donors with incorporation of two atoms of oxygen.

In a further particular embodiment, the EC 1.13.11.—enzyme is classified as EC 1.13.11.12, EC 1.13.11.31, EC 1.13.11.33, EC 1.13.11.34, EC 1.13.11.40, EC 1.13.11.44 or EC 1.13.11.45, designated lipoxygenase, arachidonate 12-lipoxygenase, arachidonate 15-lipoxygenase, arachidonate 5-lipoxygenase, arachidonate 8-lipoxygenase, linoleate diol synthase, and linoleate 1 1-lipoxygenase, respectively).

Examples of effective amounts of fatty acid oxidizing enzyme are from 0.001 to 400 U/g DS (dry solids). Preferably, the fatty acid oxidizing enzyme is used in an amount of 0.01 to 100 U/g DS, more preferably 0.05 to 50 U/g DS, and even more preferably 0.1 to 20 U/g DS. Further optimization of the amount of fatty acid oxidizing enzyme can hereafter be obtained using standard procedures known in the art.

Lipoxygenase

In a preferred embodiment the fatty acid oxidizing enzyme is a lipoxygenase (LOX), classified as EC 1.13.11.12, which is an enzyme that catalyzes the oxygenation of polyunsaturated fatty acids, especially cis,cis-1,4-dienes, e.g., linoleic acid and produces a hydroperoxide. But also other substrates may be oxidized, e.g., monounsaturated fatty acids.

Microbial lipoxygenases can be derived from, e.g., Saccharomyces cerevisiae, Thermoactinomyces vulgaris, Fusarium oxysporum, Fusarium proliferatum, Thermomyces lanuginosus, Pyricularia oryzae, and strains of Geotrichum. The preparation of a lipoxygenase derived from Gaeumannomyces graminis is described in Examples 34 of WO 02/20730. The expression in Aspergillus oryzae of a lipoxygenase derived from Magnaporthe salvinii is described in Example 2 of WO 021086114, and this enzyme can be purified using standard methods, e.g., as described in Example 4 of WO 02/20730 .

Lipoxygenase (LOX) may also be extracted from plant seeds, such as soybean, pea, chickpea, and kidney bean. Alternatively, lipoxygenase may be obtained from mammalian cells, e.g., rabbit reticulocytes.

Lipoxygenase activity may be determined as described in the “Materials & Methods” section below.

Examples of effective amounts of lipoxygenase (LOX) are from 0.001 to 400 U/g DS (dry solids). Preferably, the lipoxygenase is used in an amount of 0.01 to 100 U/g DS, more preferably 0.05 to 50 U/g DS, and even more preferably 0.1 to 20 U/g DS. Further optimization of the amount of lipoxygenase can hereafter be obtained using standard procedures known in the art.

Additional Enzymes

In a preferred embodiment of the invention one or more additional enzyme activities may be used in combination with (such as prior to, during or following) the fatty acid oxidizing enzyme treatment of the present invention. Preferred additional enzymes are esterases, such as lipases and/or phospholipases, proteases, hemicellulase or cellulase, amylases, such as alpha-amylases, maltogenic alpha-amylases, beta-amylases, or glucoamylases, or mixtures thereof.

Starch Degrading Enzyme

Any starch degrading enzyme suitable for converting the released starch to a form suitable for ethanol production may be used. Preferably, the starch degrading enzyme is a raw starch hydrolyzing enzyme, that is, an enzyme which is able to hydrolyze alpha 1,4 glucosidic linkages. Preferably the starch degrading enzyme(s) has/have the ability to hydrolyze starch under acidic conditions of below pH 7. Examples of suitable starch degrading enzymes for use in the present invention include CGTases, alpha-amylases, glucoamylases and combinations thereof.

Amylase

Preferred are alpha-amylases of fungal or bacterial origin. More preferably, the alpha-amylase is a Bacillus alpha-amylase, such as, derived from a strain of B. lichenifornis, B. amyloliquefaciens, or B. stearothermophilus. Other alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. Other alpha-amylase variants and hybrids are described in WO 96/23874, WO 97/41213, and WO 99/19467. Other alpha-amylase includes alpha-amylases derived from a strain of Aspergillus, such as, Aspergillus oryzae and Aspergillus niger alpha-amylases. In a preferred embodiment, the alpha-amylase is an acid alpha-amylase. In a more preferred embodiment the acid alpha-amylase is an acid fungal alpha-amylase or an acid bacterial apha-amylase. More preferably, the alpha-amylase is an acid fungal alpha-amyiase derived from the genus Aspergillus. In a preferred embodiment, the alpha-amylase is an acid alpha-amylase. The term “acid alpha-amylase” means an alpha-amylase (E.C. 3.2.1.1) which added in an effective amount has activity at a pH in the range of 3.0 to 7.0, preferably from 3.5 to 6.0, or more preferably from 4.0-5.0.

A preferred acid fungal alpha-amylase is a Fungamyl-like alpha-amylase. In the present disclosure, the term “Fungamyl-like alpha-amylase” indicates an alpha-amylase which exhibits a high homology, i.e., more than 50%, 55%, 60%, 65%, 70%, 75%, 80%, 85% or even 90% identity to the amino acid sequence shown in SEQ ID NO: 10 in WO 96/23874. When used as a maltose generating enzyme fungal alpha-amylases may be added in an amount of 0.001-1.0 AFAU/g DS, preferably from 0.002-0.5 AFAU/g DS, preferably 0.02-0.1 AFAU/g DS.

Preferably the alpha-amylase is an acid alpha-amylase, preferably from the genus Aspergillus, preferably of the species Aspergillus niger. In a preferred embodiment the acid fungal alpha-amylase is the one from A. niger disclosed as “AMYA_ASPNG” in the Swiss-prot/TeEMBL database under the primary accession no. P56271. Also variant of said acid fungal amylase having at least 70% homology (identity), such as at least 80%, even at least 90% identity, such as at least 95%, 96%, 97%, 98%, 99% identity thereto is contemplated.

For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10, and gap length penalty of 10. Pairwise alignment parameters were Ktuple=1, gap penalty=3, windows=5, and diago-nals=5].

Preferred bacterial acid alpha-amylases for use in the present invention may be derived from a strain of the genus Bacillus, preferably B. licheniformis, B. amyloliquefaciens, and B. stearothermophilus.

Preferred commercial compositions comprising alpha-amylase include MYCOLASE™ from DSM (Gist Brochades), BAN™, TERMAMYL™ SC, FUNGAMYL™, LIQUOZYME™ X and SANT™ SUPER, SAN™ EXTRA L (Novozymes A/S) and CLARASE™ L-40,000, DEX-LO™, SPEYME FRED, SPEZYME™ AA, and SPEZYME™ DELTA AA (Genencor Int.), and the acid fungal alpha-amylase sold under the trade name SP 288 (available from Novozymes A/S, Denmark).

The amylase may also be a maltogenic alpha-amylase. A “maltogenic alpha-amylase” (glucan 1,4-alpha-maltohydrolase, E.C. 3.2.1.133) is able to hydrolyze amylose and amylopectin to maltose in the alpha-configuration. A maltogenic alpha-amylase from B. stearothermophilus strain NCIB 11837 is commercially available from Novozymes A/S under the tradename NOVAMYL™. Maltogenic alpha-amylases are described in U.S. Pat. Nos. 4,598,048, 4,604,355 and 6,162,628, which are hereby incorporated by reference. Preferably, the maltogenic alpha-amylase is used in a raw starch hydrolysis process, as described, e.g., in WO 95/10627, which is hereby incorporated by reference.

The starch degrading enzymes are added in amounts effective to hydrolyze starch obtained from the distillers' grains, such as, in amounts from about 0.001 to 0.5% wt of solids, more preferably, from about 0.05 to 0.5% wt of solids.

The alpha-amylase may be added in amounts well-known in the art. When measured in AAU units the acid alpha-amylase activity is preferably present in an amount of 5-50,0000 MU/kg of DS, in an amount of 500-50,000 MU/kg of DS, or more preferably in an amount of 100-10,000 AAU/kg of DS, such as 500-1,000 AAU/kg DS. Fungal acid alpha-amylase are preferably added in an amount of 10-10,000 AFAU/kg of DS, in an amount of 500-2,500 AFAU/kg of DS, or more preferably in an amount of 100-1,000 AFAU/kg of DS, such as approximately 500 AFAU/kg DS.

Glucoamylase

The glucoamylase used according to an embodiment of the process of the invention may be derived from any suitable source, e.g., derived from a microorganism or a plant. Preferred glucoamylases are of fungal or bacterial origin, selected from the group consisting of Aspergillus glucoamylases, in particular A. niger G1 or G2 glucoamylase (Boel et al. (1984), EMBO J. 3 (5), p. 1097-1102), or variants thereof, such as disclosed in WO 92/00381 and WO 00/04136; the A. awamori glucoamylase (WO 84/02921), A. oryzae (Agric. Biol. Chem. (1991), 55 (4), p. 941-949), or variants or fragments thereof.

Other Aspergillus glucoamylase variants include variants to enhance the thermal stability: G137A and G139A (Chen et al. (1996), Prot. Engng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Other glucoamylases include Talaromyces glucoamylases, in particular, derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

Commercially available compositions comprising glucoamylase include AMG 200L; AMG.300 L; AMG E, SAN™ SUPER, SAN™ EXTRA L, SPIRIZYME™ PLUS, SPIRIZYME™ FUEL, SPIRIZYME™ FG, SPIRIZYME™ E, and (all from Novozymes A/S); OPTIDEX™ 300 (from Genencor Int.); AMIGASE™ and AMIGASE™ PLUS (from DSM); G-ZYME™ G900, G-ZYME™ and G990 ZR (from Genencor Int.).

Glucoamylases may in an embodiment be added in an amount of 0.02-20 AGU/g DS, preferably 0.1-10 AGU/g DS, such as 2 AGU/g DS.

The starch recovered from distillers' grains may be used for additional ethanol production, such as, by feeding the raw starch and/or the treated starch (comprising oligosaccharides) into the main ethanol process stream, such as, for liquefaction, saccharification and/or fermentation. Preferably, treated starch is fed directly into a saccharification, fermentation, SSF or LSF process for further ethanol production. Altematively, the raw starch or treated starch is fed into a liquefaction process for further ethanol production.

CGTase

Cyclomaltodextrin glucanotransferase (E.C. 2.4.1.19), also designated cyclodextrin glucanotransferase or cyclodextrin glycosyltransferase, in the following termed CGTase, catalyses the conversion of starch and similar substrates into cyclomaltodextrins via an intramolecular transglycosylation reaction, thereby forming cyclomaltodextrins (CD) of various sizes. The GCTase used according to an embodiment of the process of the invention may be derived from any suitable source, e.g., derived from a microorganism or a plant. Examples of contemplated CGTases include CGTases derived from a strain of Bacillus agardhaerans (see e.g., WO 02/06508), Bacillus autolyticus, a strain of Bacillus cereus, a strain of Bacillus circulans, a strain of Bacillus circulans var. alkalophilus, a strain of Bacillus coagulans, a strain of Bacillus firmus, a strain of Bacillus halophilus, a strain of Bacillus macerans, a strain of Bacillus megaterium, a strain of Bacillus ohbensis, a strain of Bacillus stearothermophilus, a strain of Bacillus subtilis, a strain of Klebsiella pneumonia, a strain of Thermoanaerobacter or Thermoanaerobium (see, e.g., WO 89/03421), such as strain of Thermoanaerobacter ethanolicus, a strain of Thermoanaerobacter finnii, a strain of Clostridium (see, e.g., WO 91/09962), such as a strain of Clostridium thermoamylolyticum, a strain of Clostridium thermosaccharolyticum, or a strain of Thermoanaerobacterium thermosulfurigenes. Contemplated CGTase variants include variant disclosed in WO 96/33267 and WO 99/15633 hereby incorporated by reference.

An example of a CGTases suitable for use in the present invention is TORUZYME™ (available from Novozymes A/S, Denmark).

Protease

Any protease suitable for converting the released protein to forms suitable for ethanol production may be used. The protease treated material provides nutrition to the yeast. Preferred proteases for use in the present invention have the ability to hydrolyze proteins under acidic conditions below pH 7.

Suitable proteases include fungal and bacterial proteases. Preferred proteases are acidic proteases, i.e., proteases characterized by the ability to hydrolyze proteins under acidic conditions below pH 7, preferably pH 3-7. Suitable acid fungal proteases include fungal proteases derived from Aspergillus, Mucor, Rhizopus, Candida, Coriolus, Endothia, Enthomophtra, Irpex, Penicillium, Sclerotium and Torulopsis. Especially contemplated are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae; and acidic proteases from Mucor pusillus or Mucor miehei.

Bacterial proteases, which are not acidic proteases, include the commercially available products ALCALASE™ and NEUTRASE™ (available from Novozymes A/S). Other proteases include GC106 and SPEZYME FAN (available from Genencor Int, Inc., USA and NOVOZYM™ 50006, NOVOREN™ and FLAVORZYM™ (all available from Novozymes A/S, Denmark).

Preferably, the protease is an aspartic acid protease, as described, for example, Handbook of Proteolytic Enzymes, Edited by A. J. Barrett, N. D. Rawlings and J. F. Woessner, Academic Press, San Diego, 1998, Chapter 270). Suitable examples of aspartic acid protease include, e.g., those disclosed in R. M. Berka et al. Gene, 96, 313 (1990)); (R. M. Berka et al. Gene, 125, 195-198 (1993)); and Gomi et al. Biosci. Biotech. Biochem. 57, 1095-1100 (1993), which are hereby incorporated by reference.

The proteases are added in amounts effective to convert protein obtained from the distillers' grains to oligopeptides and amino acids, such as, in amounts from about 0.001 to 0.5% wt. of solids, more preferably, about 0.05 to 0.5% wt of solids.

The recovered protein is preferably used in ethanol production, such as, by feeding the protease treated protein (comprising oligopeptides and amino acids) into the main ethanol process stream to improve fermentation efficiency by providing the yeast with improved nutritional benefits. Preferably, the protease treated protein is fed directly into the saccharification, fermentation, SSF or LSF process to further ethanol production. Altematively, the released protein or protease treated protein are fed into the liquefaction process.

Hemicellulase or Cellulase

In an embodiment of the invention distillers' grains, preferably, DDG, is treated with a fatty acid oxidizing enzyme and further a hemicellulase and/or cellulase. Any hemicellulase suitable for use in releasing the starch and non-starch components from distillers' grain may be used. Preferred hemicellulase for use in the present invention include xylanases, arabinofuranosidases, acetyl xylan esterase, glucuronidases, endo-galactanase, mannases, endo or exo arabinases, exo-galactanses, and mixtures thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes A/S, Denmark). The hemicellulase is added in an amount effective to release starch and non-starch components present in distillers' grain, such as, in amounts from about 0.001 to 0.5% wt. of solids, more preferably, from about 0.05 to 0.5% wt. of solids.

Any cellulase suitable for use in releasing the starch and non-starch components from distillers' grain may be used in the present invention. The cellulase activity used according to the invention may be derived from any suitable origin, preferably, the cellulase is of microbial origin, such as derivable from a strain of a filamentous fungus (e.g., Aspergillus, Trichoderma, Humicola, Fusarium). Preferably, the cellulase composition acts on both cellulosic and lignocellulosic material. Preferred cellulases for use in the present invention include exo-acting celluases and cellobiases, and combinations thereof. More preferably, the treatment involves the combination of an exo-acting cellulase and a cellobiase. Preferably, the cellulases have the ability to hydrolyze cellulose or lignocellulose under acidic conditions of below pH 7. Examples of cellulases suitable for use in the present invention include, for example, CELLULCLAST™ (available from Novozymes A/S), NOVOZYM™ 188 (available from Novozymes A/S) Other commercially available preparations comprising cellulase which may be used include CELLUZYME™, CEREFLO™ and ULTRAFLO™ (Novozymes A/S), LAMINEX™ and SPEZYME™ CP (Genencor Int.) and ROHAMENT™ 7069 W (from Röhm GmbH). The cellulase enzymes are added in amounts effective to release starch and/or non-starch components present in distillers' grain, such as, in amounts from about 0.001 to 0.5% wt. of solids, more preferably, 0.05% to 0.5% wt. of solids.

Esterases

In a preferred embodiment of the invention the fatty acid oxidizing enzyme treatment is carried out together with an esterase treatment. The enzymes may be added prior to or together with the fatty acid oxidizing enzyme.

As used herein, an “esterase” also referred to as a carboxylic ester hydrolyases, refers to enzymes acting on ester bonds, and includes enzymes classified in EC 3.1.1 Carboxylic Ester Hydrolases according to Enzyme Nomenclature (available at http://www.chem.qmw.ac.uk/iubmb/enzyme or from Enzyme Nomenclature 1992, Academic Press, San Diego, Calif., with Supplement 1 (1993), Supplement 2 (1994), Supplement 3 (1995), Supplement 4 (1997) and Supplement 5, in Eur. J. Biochem. 1994, 223, 1-5; Eur. J. Biochem. 1995, 232, 1-6; Eur. J. Biochem. 1996, 237, 1-5; Eur. J. Biochem. 1997, 250; 1-6, and Eur. J. Biochem. 1999, 264, 610-650; respectively). Non-limiting examples of esterases include arylesterase, triacylglycerol lipase, acetylesterase, acetylcholinesterase, cholinesterase, tropinesterase. pectinesterase, sterol esterase, chlorophyllase, L-arabinonolactonase, gluconolactonase, uronolactonase, tannase, retinyl-palmitate esterase, hydroxybutyrate-dimer hydrolase, acylglycerol lipase, 3-oxoadipate enol-lactonase, 1,4-lactonase, galactolipase, 4-pyridoxolactonase, acylcamitine hydrolase, aminoacyl-tRNA hydrolase, D-arabinonolactonase, 6-phosphogluconolactonase, phospholipase A1, 6-acetylglucose deacetylase, lipoprotein lipase, dihydrocoumarin lipase, limonin-D-ring-lactonase, steroid-lactonase, triacetate-lactonase, actinomycin lactonase, orsellinate-depside hydrolase, cephalosporin-C deacetylase, chlorogenate hydrolase, alpha-amino-acid esterase, 4-methyloxaloacetate esterase, carboxymethylenebutenolidase, deoxylimonate A-ring-lactonase, 2-acetyl-1-alkylglycerophosphocholine esterase, fusarinine-C ornithinesterase, sinapine esterase, wax-ester hydrolase, phorbol-diester hydrolase, phosphatidylinositol deacylase, sialate O-acetylesterase, acetoxybutynylbithiophene deacetylase, acetylsalicylate deacetylase, methylumbelliferyl-acetate deacetylase, 2-pyrone-4,6-dicarboxylate lactonase, N-acetylgalactosaminoglycan deacetylase, juvenile-hormone esterase, bis(2-ethylhexyl)phthalate esterase, protein-glutamate methylesterase, 11-cis-retinyl-palmitate hydrolase, all-trans-retinyl-palmitate hydrolase, L-rhamnono-1,4-lactonase, 5-(3,4-diacetoxybut-1-ynyl)-2,2′-bithiophene deacetylase, fatty-acyl-ethyl-ester synthase, xylono-1,4-lactonase, N-acetylglucosaminylphosphatidylinositol deacetylase, cetraxate benzylesterase, acetylalkylglycerol acetylhydrolase, and acetylxylan esterase.

Preferred esterases for use in the present invention are lipolytic enzymes, such as, lipases (as classified by EC 3.1.1.3, EC 3.1.1.23 and/or EC 3.1.1.26) and phospholipases (as classified by EC 3.1.1.4 and/or EC 3.1.1.32, including lysophospholipases as classified by EC 3.1.1.5). Other preferred esterases are cutinases (as classified by EC 3.1.1.74).

When used in combination with processes or treatments which employ other enzymes, beside the fatty acid oxidizing enzyme, such as, amylases and glucoamylases compositions which do not inhibit these other enzymes are preferred, e.g., esterases which do not contain or contain only minor amounts of calcium-binding compounds are preferred. Similarly, esterases which do not inhibit fermentation processes are preferred, e.g., esterases which do not contain or which contain only minor amounts of glycerol are preferred.

The esterase may be added in an effective amount. Examples of such effective amounts indude from 0.01 to 400 LU/g DS (Dry Solids). Preferably, the esterase is used in an amount of 0.1 to 100 LU/g DS, more preferably 0.5 to 50 LU/g DS, and even more preferably 1 to 20 LU/g DS. Further optimization of the amount of esterase can hereafter be obtained using standard procedures known in the art.

Lipolytic Enzyme

In a preferred embodiment the esterase is a lipolytic enzyme, more preferably, a lipase. As used herein, a “lipolytic enzymes” refers to lipases and phospholipases (including lyso-phospholipases). The lipolytic enzyme is preferably of microbial origin, in particular of bacterial, fungal or yeast origin. The lipolytic enzyme used may be derived from any source, including, for example, a strain of Absidia, in particular Absidia blakesleena and Absidia corymbifera, a strain of Achromobacter, in particular Achromobacter iophagus, a strain of Aeromonas, a strain of Alternaria, in particular Afternaria brassiciola, a strain of Aspergillus, in particular Aspergillus niger and Aspergillus flavus, a strain of Achromobacter, in particular Achromobacter iophagus, a strain of Aureobasidium, in particular Aureobasidium pullulans, a strain of Bacillus, in particular Bacillus pumilus, Bacillus strearothermophilus and Bacillus subtilis, a strain of Beauveria, a strain of Brochothrix, in particular Brochothrix thermosohata, a strain of Candida, in particular Candida cylindracea (Candida rugosa), Candida paralipolytica, and Candida antarctica, a strain of Chromobacter, in particular Chromobacter viscosum, a strain of Coprinus, in particular Coprinus cinerius, a strain of Fusarium, in particular Fusarium oxysporum, Fusarium solani, Fusarium solani pisi, and Fusarium roseum culmorum, a strain of Geotricum, in particular Geotricum penicillatum, a strain of Hansenula, in particular Hansenula anomala, a strain of Humicola, in particular Humicola brevispora, Humicola brevis var. thermoidea, and Humicola insolens, a strain of Hyphozyma, a strain of Lactobacillus, in particular Lactobacillus curvatus, a strain of Metarhizium, a strain of Mucor, a strain of Paecilomyces, a strain of Penicillium, in particular Penicillium cyclopium, Penicillium crustosum and Penicilium expansum, a strain of Pseudomonas in particular Pseudomonas aeruginosa, Pseudomonas alcaligenes, Pseudomonas cepacia (syn. Burkholderia cepacia), Pseudomonas fluorescens, Pseudomonas fragi, Pseudomonas maltophilia, Pseudomonas mendocina, Pseudomonas mephitica lipolytica, Pseudomonas alcaligenes, Pseudomonas plantari, Pseudomonas pseudoalcaligenes, Pseudomonas putida, Pseudomonas stutzeri, and Pseudomonas wisconsinensis, a strain of Rhizoctonia, in particular Rhizoctonia solani, a strain of Rhizomucor, in particular Rhizomucor miehei, a strain of Rhizopus, in particular Rhizopus japonicus, Rhizopus microsporus and Rhizopus nodosus, a strain of Rhodosporidium, in particular Rhodosporidium toruloides, a strain of Rhodotorula, in particular Rhodotorula glutinis, a strain of Sporobolomyces, in particular Sporobolomyces shibatanus, a strain of Thermomyces, in particular Thermomyces lanuginosus (formerly Humicola lanuginosa), a strain of Thiarosporella, in particular Thiarosporella phaseolina, a strain of Trichoderma, in particular Trichoderma harzianum, and Trichoderma reesei, and/or a strain of Verticillium.

In a preferred embodiment, the lipolytic enzyme is derived from a strain of Aspergillus, a strain of Achromobacter, a strain of Bacillus, a strain of Candida, a strain of Chromobacter, a strain of Fusarium, a strain of Humicola, a strain of Hyphozyma, a strain of Pseudomonas, a strain of Rhizomucor, a strain of Rhizopus, or a strain of Thermomyces.

Lipase

In more preferred embodiments, the lipolytic enzyme is a lipase. Lipases may be applied herein for their ability to modify the structure and composition of triglyceride oils and fats in the fermentation media (including fermentation yeast), for example, resulting from a corn substrate. Lipases catalyze different types of triglyceride conversions, such as hydrolysis, esterification and transesterification. Suitable lipases indude acidic, neutral and basic lipases, as are well-known in the art, although acidic lipases (such as, e.g., the lipase G AMANO 50, available from Amano) appear to be more effective at lower concentrations of lipase as compared to either neutral or basic lipases. Preferred lipases for use in the present invention included Candida antarcitca lipase and Candida cylindracea lipase. More preferred lipases are purified lipases such as Candida antarcitca lipase (lipase A), Candida antarcitca lipase (lipase B), Candida cylindracea lipase, and Penicillium camembertii lipase.

The lipase the one disclosed in EP 258,068-A or may be a lipase variant such as a variant disclosed in WO 00/60063 or WO 00/32758 which is hereby incorporated by refernce Preferred commercial lipases include LECITASE™, LIPOLASE™ and LIPEX™ (available from Novozymes A/S, Denmark) and G AMANO 50 (available from Amano).

Lipases are preferably added in amounts from about 1 to 400 LU/g DS, preferably 1 to 10 LU/g DS, and more preferably 1 to 5 LU/g DS.

Cutinase

In another preferred embodiment of the present invention, the at least one esterase is a cutinase. Cutinases are enzymes which are able to degrade cutin. The cutinase may be derived from any source. In a preferred embodiment, the cutinase is derived from a strain of Aspergillus, in particular Aspergillus oryzae, a strain of Altemara, in particular Alternaria brassiciola, a strain of Fusarium, in particular Fusarum solani, Fusadum solani pisi, Fusadum roseum culmorum, or Fusarium roseum sambucium, a strain of Helminthosporum, in particular Helminthosporum sativum, a strain of Humicola, in particular Humicola insolens, a strain of Pseudomonas, in particular Pseudomonas mendocina, or Pseudomonas putida, a strain of Rhizoctonia, in particular Rhizoctonia solani, a strain of Streptomyces, in particular Streptomyces scabies, or a strain of Ulocladium, in particular Ulocladium consortiale. In a most preferred embodiment the cutinase is derived from a strain of Humicola insolens, in particular the strain Humicola insolens DSM 1800. Humicola insolens cutinase is described in WO 96/13580 which is herby incorporated by reference. The cutinase may be a variant such as one of the variants disclosed in WO 00/34450 and WO 01/92502 which is hereby incorporated by reference. Preferred cutinase variants include variants listed in Example 2 of WO 01/92502 which are hereby specifically incorporated by reference. An effective amount of cutinase is between 0.01 and 400 LU/g DS, preferably from about 0.1 to 100 LU/g DS, more preferably, 1 to 50 LU/g DS. Further optimization of the amount of cutinase can hereafter be obtained using standard procedures known in the art.

Phospholipase

In another preferred embodiment, the at least one esterase is a phospholipase. As used herein, the term phospholipase is an enzyme which has activity towards phospholipids. Phospholipids, such as lecithin or phosphatidylcholine, consist of glycerol esterified with two fatty acids in an outer (sn-1) and the middle (sn-2) positions and esterified with phosphoric acid in the third position; the phosphoric acid, in turn, may be esterified to an amino-alcohol. Phospholipases are enzymes which participate in the hydrolysis of phospholipids. Several types of phospholipase activity can be distinguished, including phospholipases A₁ and A₂ which hydrolyze one fatty acyl group (in the sn-1 and sn-2 position, respectively) to form lysophospholipid; and lysophospholipase (or phospholipase B) which can hydrolyze the remaining fatty acyl group in lysophospholipid. Phospholipase C and phospholipase D (phosphodiesterases) release diacyl glycerol or phosphatidic acid respectively.

The term phospholipase includes enzymes with phospholipase activity, e.g., phospholipase A (A₁ or A₂), phospholipase B activity, phospholipase C activity or phospholipase D activity. The term “phospholipase A” used herein in connection with an enzyme of the invention is intended to cover an enzyme with Phospholipase A₁ and/or Phospholipase A₂ activity. The phospholipase activity may be provided by enzymes having other activities as well, such as, e.g., a lipase with phospholipase activity. The phospholipase activity may, e.g., be from a lipase with phospholipase side activity. In other embodiments of the invention the phospholipase enzyme activity is provided by an enzyme having essentially only phospholipase activity and wherein the phospholipase enzyme activity is not a side activity.

The phospholipase may be of any origin, e.g., of animal origin (such as, e.g., mammalian), e.g., from pancreas (e.g., bovine or porcine pancreas), or snake venom or bee venom. Alternatively, the phospholipase may be of microbial origin, e.g. from filamentous fungi, yeast or bacteria, such as the genus or species Aspergillus, e.g., A. niger, Dictyostelium, e.g. D. discoideum; Mucor, e.g. M. javanicus, M. mucedo, M. subtilissimus; Neurospora, e.g. N. crassa; Rhizomucor, e.g., R. pusillus; Rhizopus, e.g. R. arrhizus, R. japonicus, R. stolonifer; Sclerotinia, e.g., S. libertiana; Trichophyton, e.g. T rubrum; Whetzelinia, e.g. W sclerotiorum; Bacillus, e.g., B. megaterium, B. subtilis; Citrobacter, e.g., C. freundii; Enterobacter, e.g., E. aerogenes, E. cloacae Edwardsiella, E. tarda; Erwinia, e.g., E. herbicola; Escherichia, e.g., E. coli; Klebsiella, e.g., K. pneumoniae; Proteus, e.g., P. vulgaris; Providencia, e.g. P. stuartii; Salmonella, e.g. S. typhimurium; Serratia, e.g., S. liquefasciens, S. marcescens; Shigella, e.g., S. flexneri; Streptomyces, e.g., S. violeceoruber, Yersinia, e.g., Y. enterocolitica. Thus, the phospholipase may be fungal, e.g., from the class Pyrenomycetes, such as the genus Fusarium, such as a strain of F. culmorum, F. heterosporum, F. solani, or a strain of F. oxysporum. The phospholipase may also be from a filamentous fungus strain within the genus Aspergillus, such as a strain of Aspergillus awamori, Aspergillus foetidus, Aspergillus japonicus, Aspergillus niger or Aspergillus oryzae. Preferred commercial phospholipases include LECITASE™ and LECITASE™ ULTRA (available from Novozymes A/S, Denmark).

An effective amount of phospholipase is between 0.01 and 400 LU/g DS, preferably from about 0.1 to 100 LU/g DS, more preferably, 1 to 50 LU/g DS. Further optimization of the amount of phospholipase can hereafter be obtained using standard procedures known in the art.

Chemical and/or Mechanical Treatment

In preferred embodiments of the present invention, chemical treatment and/or mechanical treatment processes are used in combination with the enzymatic processes described herein to promote the release of starch and non-starch components from distillers' grain, that is, to further the release of the starch and non-starch components from distillerS' grain or to further the enzymatic processes described herein. Preferably, the chemical and/or mechanical treatment processes are carried out prior to the enzymatic processes in a pre-treatment process so as to improve the enzymatic processes described herein. In the alternative, the chemical and/or mechanical treatment processes are carried out simultaneously with the enzymatic processes, such as simultaneously with the fatty acid oxidizing enzyme treatment described herein.

As used in the present invention, “a chemical treatment process” refers to any chemical treatment process which can be used to promote the release of starch and/or non-starch components, in particular, protein, from distillers' grains. Examples of chemical treatments suitable for use in the present invention include, for example, acid treatment, wet oxidation, and solvent treatment. More preferably, the chemical treatment process is an acid treatment process, more preferably, a continuous dilute or mild acid treatment, such as, treatment with sulfuric acid, or another organic acid, such as acetic acid, citric acid, tartaric acid, succinic acid, or mixtures thereof. Other adds may also be used. Mild acid treatment means in the context of the invention that the treatment pH lies in the range from 1 to 5, preferably 1 to 3. In a specific embodiment the acid concentration is in the range from 0.5 to 1.7 wt % sulfuric acid. Wet oxidation techniques involve the use of oxidizing agents, such as; sulfite based oxidizing agents and the like. Examples of solvent treatments include treatment with DMSO (Dimethyl Sulfoxide) and the like. Chemical treatment processes are generally carried out for about 5 to about 10 minutes, but may be carried out for shorter or longer periods of time.

As used in the present invention, the phrase “a mechanical treatment process” refers to any mechanical treatment process which can be used to promote the release of starch and non-starch components, in particular protein, from distillers' grains, in particular from DDG. Preferably, a mechanical treatment process involves a process which uses high pressure and high temperature to promote the release of the starch and non-starch components in distillers' grains. In context the in invention high pressure means pressure in the range from 300 to 600, preferably 400 to 500, such as around 450 psi. In context the in invention high temperature means pressure in the range from about 100 to 300° C., preferably from about 140 to 235° C. In a specific embodiment the impregnation is carried out at a pressure of about 450 psi and at a temperature of about 235° C. More preferably, the mechanical process is a batch-process, steam gun hydrolyzer system which uses high pressure and high temperature, such as, using the Sunds Hydrolyzer (available from Sunds Defibrator AB (Sweden).

In preferred embodiments, both chemical and mechanical treatment is carried out, for example, involving, for example, both mild acid treatment and high temperature and pressure treatment. The chemical and mechanical processes may be carried out sequentially or simultaneously, as desired.

Accordingly, in a more preferred embodiment, the process comprises the steps of (a) pre-treating distillers' grain, preferably, DDG, with a chemical treatment and/or a mechanical treatment to promote the release of starch and protein present in distillers' grain, (b) treating the chemically and/or mechanically treated distillers' grain with a fatty acid oxidizing enzyme to further promote the release of starch and/or protein from distillers' grain and (c) treating the released starch and/or protein with a starch degrading enzyme and/or a protease. Preferably, the process further comprises the step of feeding the treated starch and/or protein into the ethanol process stream, more preferably at the liquefaction, saccharification and/or fermentation steps, and most preferably at the saccharification and/or fermentation steps, such as, an SSF process or LSF process.

The enzymatic treatment is carried out in a suitable aqueous environment, which can be readily determined by one skilled in the art practicing the present invention. Any suitable process time, holding time, temperature and pH conditions may be employed, which can be readily determined by one skilled in the art practicing the present invention. Preferably, the enzymatic treatment processes is from about 2 to about 60 hours. The temperature of the enzymatic treatment processes are preferably from about 40 to 60° C. The pH of aqueous treatment solution used in the enzymatic treatment process is about preferably from about 4 to about 7, more preferably 4 to 5.

Various modifications of the invention described herein will become apparent to those skilled in the art. Such modifications are intended to fall within the scope of the appending claims.

### MATERIALS & METHODS

Materials

Enzymes:

Fatty acid-oxidizing enzyme: Lipoxygenase derived from Magnaporthe salvini, disclosed in WO 02/086114 (available from Novozymes A/S, Denmark).
Bacteriai Alpha-Amylase A: Bacillus stearothermophilus alpha-amylase variant with the mutations: I181*+G182*+N193F disclosed in U.S. Pat. No. 6,187,576 and available on request from Novozymes ANS, Denmark.
Glucoamylase-SF: Balanced blend of Aspergillus niger glucoamylase and A. niger acid alpha-amylase having a ratio between AGU and AFAU of approx. 9:1.
Protease: ALCASE™ 2.4L FG available from Novozymes A/S, Denmark.
Yeast: Ethanol Red available from Red Star/Lesaffre, USA.
   Methods:
   Lipoxygenase Activity

Lipoxygenase activity may be determined spectrophotometrically at 25° C. by monitoring the formation of hydroperoxides. For the standard analysis, 10 microliters enzyme is added to a 1 ml quartz cuvette containing 980 micro liter 25 mM sodium phosphate buffer (pH 7.0) and 10 micro liter of substrate solution (10 mM linoleic acid dispersed with 0.2% (v/v) Tween20 (should not be kept for extended time periods)). The enzyme is typically diluted sufficiently to ensure a turn-over of maximally 10% of the added substrate within the first minute. The absorbance at 234 nm is followed and the rate is estimated from the linear part of the curve. The cis-trans-conjugated hydro(pero)xy fatty acids are assumed to have a molecular extinction coefficient of 23,000 M⁻cm⁻.

Alpha-amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C. +/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

Determination of FAU Activity

One Fungal Alpha-Amylase Unit (FAU) is defined as the amount of enzyme, which breaks down 5.26 g starch (Merck Amylum solubile Erg. B.6, Batch 9947275) per hour based upon the following standard conditions:

| Substrate | Soluble starch | |
|---|---|---|
| Temperature | 37° | C. |
| pH | 4.7 | |
| Reaction time | 7-20 | minutes |

Determination of Acid Alpha-amylase Activity (AFAU)

Acid alpha-amylase activity is measured in AFAU Acid Eungal Alpha-amylase Units), which are determined relative to an enzyme standard.

The standard used is AMG 300 L (from Novozymes A/S, Denmark, glucoamylase wildtype Aspergillus niger G1, also disclosed in Boel et al. (1984), EMBO J. 3 (5), p. 1097-1102) and WO 92/00381). The neutral alpha-amylase in this AMG falls after storage at room temperature for 3 weeks from approx. 1 FAU/mL to below 0.05 FAU/mL.

The acid alpha-amylase activity in this AMG standard is determined in accordance with the following description. In this method, 1 AFAU is defined as the amount of enzyme, which degrades 5.260 mg starch dry matter per hour under standard conditions.

Iodine forms a blue complex with starch but not with its degradation products. The intensity of color is therefore directly proportional to the concentration of starch. Amylase activity is determined using reverse colorimetry as a reduction in the concentration of starch under specified analytic conditions.

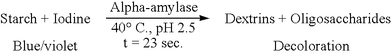

Standard Conditions/reaction Conditions: (Per Minute)

Substrate: Starch, approx. 0.17 g/L

Buffer: Citate, approx. 0.03 M

Iodine (I₂): 0.03 g/L

CaCl₂: 1.85 mM

pH: 2.50±0.05

Incubation temperature: 40° C.

Reaction time: 23 seconds

Wavelength: lambda=590nm

Enzyme concentration: 0.025 AFAU/mL

Enzyme working range: 0.01-0.04 AFAU/mL

If further details are preferred these can be found in EB-SM-0259.02/01 available on request from Novozymes A/S, Denmark, and incorporated by reference.

Acid Alpha-amylase Units (AAU)

The acid alpha-amylase activity can be measured in MU (Acid Alpha-amylase Units), which is an absolute method. One Acid Amylase Unit (MU) is the quantity of enzyme converting 1 g of starch (100% of dry matter) per hour under standardized conditions into a product having a transmission at 620 nm after reaction with an iodine solution of known strength equal to the one of a color reference.

Standard Conditions/Reaction Conditions:

Substrate: Soluble starch. Concentration approx. 20 g DS/L.
Buffer: Citrate, approx. 0.13 M, pH=4.2
Iodine solution: 40.176 g potassium iodide +0.088 g iodine/L
City water 15°-20°dH (German degree hardness)
pH: 4.2
Incubation temperature: 30° C.
Reaction time: 11 minutes
Wavelength: 620 nm
Enzyme concentration: 0.13-0.19 AAU/mL
Enzyme working range: 0.13-0.19 AAU/mL

The starch should be Lintner starch, which is a thin-boiling starch used in the laboratory as colorimetric indicator. Lintner starch is obtained by dilute hydrochloric acid treatment of native starch so that it retains the ability to color blue with iodine. Further details can be found in EP 0140410B2, which disclosure is hereby included by reference.

Glucoamylase Activity (AGI)

Glucoamylase (equivalent to amyloglucosidase) converts starch into glucose. The amount of glucose is determined here by the glucose oxidase method for the activity determination. The method described in the section 76-11 Starch-Glucoamylase Method with Subsequent Measurement of Glucose with Glucose Oxidase in “Approved methods of the American Association of Cereai Chemists”. Vol. 1-2 AACC, from American Association of Cereal Chemists, (2000); ISBN: 1-891127-12-8.

One glucoamylase unit (AGI) is the quantity of enzyme which will form 1 micromol of glucose per minute under the standard conditions of the method.

Standard Conditions/Reaction Conditions:

Substrate: Soluble starch.
Concentration approx. 16 g dry matter/L.
Buffer: Acetate, approx. 0.04 M, pH=4.3
pH: 4.3
Incubation temperature: 60° C.
Reaction time: 15 minutes
Termination of the reaction: NaOH to a concentration of approximately 0.2 g/L (pH˜9)
Enzyme concentration: 0.15-0.55 AAU/mL.

The starch should be Lintner starch, which is a thin-boiling starch used in the laboratory as colorimetric indicator. Lintner starch is obtained by dilute hydrochloric acid treatment of native starch so that it retains the ability to color blue with iodine.

Glucoamylase Activity (AGU)

The Novo Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

An autoanalyzer system may be used. Mutarotase is added to the glucose dehydrogenase reagent so that any alpha-D-glucose present is turned into beta-D-glucose. Glucose dehydrogenase reacts specifically with beta-D-glucose in the reaction mentioned above, forming NADH which is determined using a photometer at 340 nm as a measure of the original glucose concentration.

| AMG incubation: | | |
|---|---|---|
| Substrate: | maltose 23.2 mM | |
| Buffer: | acetate 0.1 M | |
| pH: | 4.30 ± 0.05 | |
| Incubation temperature: | 37° C. ± 1 | |
| Reaction time: | 5 | minutes |
| Enzyme working range: | 0.5-4.0 | AGU/mL |
| Color reaction: | | |
| GlucDH: | 430 | U/L |
| Mutarotase: | 9 | U/L |
| NAD: | 0.21 | mM |
| Buffer: | phosphate 0.12 M; 0.15 M NaCl | |
| pH: | 7.60 ± 0.05 | |
| Incubation temperature: | 37° C. ± 1 | |
| Reaction time: | 5 | minutes |
| Wavelength: | 340 | nm |

A folder (EB-SM-0131.02/01) describing this analytical method in more detail is available on request from Novozymes A/S, Denmark, which folder is hereby included by reference.

Cutinase Activity (LU)

The cutinase activity is determined as lipolytic activity determined using tributyrine as substrate. This method was based on the hydrolysis of tributyrin by the enzyme, and the alkali consumption is registered as a function of time. One Lipase Unit (LU) is defined as the amount of enzyme which, under standard conditions (i.e. at 30.0 degree celsius; pH 7.0; with Gum Arabic as emulsifier and tributyrine as substrate) liberates 1 micro mol titrable butyric acid per minute. A folder AF 95/5 describing this analytical method in more detail is available upon request from Novozymes A/S, Denmark, which folder is hereby included by reference.

Xylanolytic Activity (FXU)

The xylanolytic activity can be expressed in FXU-units, determined at pH 6.0 with remazol-xylan (4-O-methyl-D-glucurono-D-xylan dyed with Remazol Brilliant Blue R, Fluka) as substrate.

A xylanase sample is incubated with the remazol-xylan substrate. The background of non-degraded dyed substrate is precipitated by ethanol. The remaining blue color in the supematant (as determined spectrophotometrically at 585 nm) is proportional to the xylanase activity, and the xylanase units are then determined relatively to an enzyme standard at standard reaction conditions, i.e. at 50.0° C., pH 6.0, and 30 minutes reaction time.

A folder EB-SM-352.02101 describing this analytical method in more detail is available upon request to Novozymes A/S, Denmark, which folder is hereby induded by reference.

Cellulytic Activity (EGU)

The cellulytic activity may be measured in endo-glucanase units (EGU), determined at pH 6.0 with carboxymethyl cellulose (CMC) as substrate. A substrate solution is prepared, containing 34.0 g/l CMC (Hercules 7 LFD) in 0.1 M phosphate buffer at pH 6.0. The enzyme sample to be analyzed is dissolved in the same buffer. 5 ml substrate solution and 0.15 ml enzyme solution are mixed and transferred to a vibration viscosimeter (e.g. MIVI 3000 from Sofraser, France), thermostated at 40° C. for 30 minutes. One EGU is defined as the amount of enzyme that reduces the viscosity to one half under these conditions. The amount of enzyme sample should be adjusted to provide 0.01-0.02 EGU/ml in the reaction mixture. The arch standard is defined as 880 EGU/g.

A folder EB-SM-0275.02/01 describing this analytical method in more detail is available upon request to Novozymes A/S, Denmark, which folder is hereby included by reference.

RS Determination: Reducing Sugar Assay: PHBAH

1. Prepare glucose standards from a stock 2 mg/mL (10 mM) solution. Dilute in Carbonate/Bicarbonate buffer to give standards of 0.1, 0.075, 0.05, 0.0375, 0.025, 0.0125 and 0.0075 mg/mL. Standards can be aliquotted and frozen at 40° C. for up to 3 weeks.
2. Dilute samples 1:10 in Carbonate/Bicarbonate by adding 30 μL of sample to 270 microL of buffer. Mix. Additional dilutions are made serially from the initial 1:10 dilution into Carbonate/Bicarbonate as necessary to get the test response to fall on the standard curve.
3. Into the wells of a 0.2 mL PCR plate (Greiner), transfer 100 μL of each sample, standard and blank. Standards are analyzed in duplicate, samples in triplicate.
4. Add 50 microL of PHBAH reagent to each well. Seal plate with a Mylar sealer.
5. Place the PCR plate in a 90° C. heat block for 10 minutes.
6. Retrieve plate and allow cooling to ambient temperature.
7. Transfer 50 microL of each sample, standard and blank to a corresponding well of a 96 well assay plate (Nunc) containing 50 microL of PHBAH buffer (1:2 dilution).
8. R-Read absorbance at 405 nm.
   Measurement of Cellulase Activity Using Filter Paper Assay (FPU Assay)
   1. Source of Method
1.1 The method is disclosed in a document entitled “Measurement of Cellulase Activities” by Adney, B. and Baker, J., 1996. Laboratory Analytical Procedure, LAP-006, National Renewabl Energy Laboratory (NREL). It is based on the IUPAC method for measuring cellulase activity (Ghose, T. K., Measurement of Cellulse Activities, Pure & Appl. Chem. 59, pp. 257-268, 1987.
   2. Procedure
2.1 The method is carried out as described by Adney and Baker, 1996, supra, except for the use of a 96 well plates to read the absorbance values after color development, as described below.
   2.2 Enzyme Assay Tubes:
2.2.1 A rolled filter paper strip (#1 Whatman; 1×6 cm; 50 mg) is added to the bottom of a test tube (13×100 mm).
2.2.2 To the tube is added 1.0 mL of 0.05 M Na-citrate buffer (pH 4.80).
2.2.3 The tubes containing filter paper and buffer are incubated 5 min. at 50° C. (±0.1° C.) in a circulating water bath.
2.2.4 Following incubation, 0.5 mL of enzyme dilution in citrate buffer is added to the tube. Enzyme dilutions are designed to produce values slightly above and below the target value of 2.0 mg glucose.
2.2.5 The tube contents are mixed by gently vortexing for 3 seconds.
2.2.6 After vortexing, the tubes are incubated for 60 mins. at 50° C. (±0.1° C.) in a circulating water bath.
2.2.7 Immediately following the 60 min. incubation, the tubes are removed from the water bath, and 3.0 mL of DNS reagent is added to each tube to stop the reaction. The tubes are vortexed 3 seconds to mix.
   2.3 Blank and Controls
2.3.1 A reagent blank is prepared by adding 1.5 mL of citrate buffer to a test tube.
2.3.2 A substrate control is prepared by placing a rolled filter paper strip into the bottom of a test tube, and adding 1.5 mL of citrate buffer.
2.3.3 Enzyme controls are prepared for each enzyme dilution by mixing 1.0 mL of citrate buffer with 0.5 mL of the appropriate enzyme dilution.
2.3.4 The reagent blank, substrate control, and enzyme controls are assayed in the same manner as the enzyme assay tubes, and done along with them.
   24 Glucose Standards
2.4.1 A 100 mL stock solution of glucose (10.0 mg/mL) is prepared, and 5 mL aliquots are frozen. Prior to use, aliquots are thawed and vortexed to mix.
2.4.2 Dilutions of the stock solution are made in citrate buffer as follows:
G1=1.0 mL stock+0.5 mL buffer=6.7 mg/mL=3.3 mg/0.5 mL
G2=0.75 mL stock+0.75 mL buffer=5.0 mg/mL=2.5 mg/0.5 mL
G3=0.5 mL stock+1.0 mL buffer=3.3 mg/mL=1.7 mg/0.5 mL
G4=0.2 mL stock+0.8 mL buffer=2.0 mg/mL=1.0 mg/0.5 mL
2.4.3 Glucose standard tubes are prepared by adding 0.5 mL of each dilution to 1.0 mL of citrate buffer.
2.4.4 The glucose standard tubes are assayed in the same manner as the enzyme assay tubes, and done along with them.
   2.5 Color Development
2.5.1 Following the 60 min. incubation and addition of DNS, the tubes are all boiled together for 5 mins. in a water bath.
2.5.2 After boiling, they are immediately cooled in an ice/water bath.
2.5.3 When cool, the tubes are briefly vortexed, and the pulp is allowed to settle. Then each tube is diluted by adding 50 microL from the tube to 200 microL of ddH20 in a 96-well plate. Each well is mixed, and the absorbance is read at 540 nm.
   2.6 Calculations (Examples are Given in the NREL Document)
2.6.1 A glucose standard curve is prepared by graphing glucose concentration (mg/0.5 mL) for the four standards (G1-G4) vs. A₅₄₀. This is fitted using a linear regression (Prism Software), and the equation for the line is used to determine the glucose produced for each of the enzyme assay tubes.
2.6.2 A plot of glucose produced (mg/0.5 mL) vs. total enzyme dilution is prepared, with the Y-axis (enzyme dilution) being on a log scale.
2.6.3 A line is drawn between the enzyme dilution that produced just above 2.0 mg glucose and the dilution that produced just below that. From this line, it is determined the enzyme dilution that would have produced exactly 2.0 mg of glucose.
2.6.4 The Filter Paper Units/mL (FPU/mL) are calculated as follows:
FPU/mL=0.37/ enzyme dilution producing 2.0 mg glucose
   Determination of DE (Dextrose Equivalent)

The DE value is measured using Fehlings liquid by forming a copper complex with the starch using pure glucose as a reference, which subsequently is quantified through iodometric titration. DE (dextrose equivalent) is defined as the amount of reducing carbohydrate (measured as dextrose-equivalents) in a sample expressed as w/w % of the total amount of dissolved dry matter. It may also be measured by the neocuproine assay (Dygert, Li Floridana(1965) Anal. Biochem. No 368). The principle of the neocuproine assay is that CuSO₄ is added to the sample, Cu²⁺ is reduced by the reducing sugar and the formed neocuproine complex is measured at 450 nm.

### EXAMPLES

### Example 1

Measurement of the Activity of Fatty Acid Oxidizing Enzymes on Linoleic Acid

An “Oxi 3000 Oximeter” (WTW, Weilheim, Germany) with a TriOxmatic 300 oxygen electrode and a standard reaction volume of 4 ml was used.

10 mg linoleic acid (10 ml 60% linoleic acid) was dissolved in 1 ml ethanol, and 2 microliters Tween 20 was added. From this stock substrate solution 50 micro liter was added into a reaction beaker containing 3.85 ml buffer solution (Britton-Robinson: 100 mM of Phosphoric-, Acetic- and Boric acid; pH adjusted with NaOH) with a small stir bar allowing solution to be mixed well, and the oxygen electrode was inserted into the reaction beaker. 100 micro liter purified enzyme solution was added, viz. (a) lipoxygenase derived from Magnaporthe salvinii at a concentration of approx. 0.4 mg/ml; or (b) lipoxygenase derived from Gaeumannomyces. graminis at a concentration of approx. 0.76 mg/ml (which means approximately 0.02 mg/ml in the final reaction). These lipoxygenases were prepared as previously described. The temperature was 25° C. The concentration of dissolved oxygen (mg/l) is measured and plotted as a function of time (min.). The enzymatic activity is calculated as the slope of the linear part of the curve (mg/l/min.) after addition of the enzyme. The baseline was corrected by subtraction when relevant, meaning that if the curve showing oxygen concentration as a function of time had a slope of above about 0.05 mg oxygen/ml/min before addition of the fatty acid oxidizing enzyme (i.e. the control), this value was subtracted from the sample slope value.

Table 1 below shows the results of the experiments.

TABLE 1

Fatty Acid Oxidizing Enzyme

| | (a) LOX from M. salvinii | (b) LOX from G. graminis |
|---|---|---|
| pH | mgO₂/ml/min | mgO₂/ml/min |
| 2 | 0.0 | 0.0 |
| 4 | 0.4 | 0.1 |
| 5 | 0.7 | 0.4 |
| 6 | 1.1 | 0.4 |
| 7 | 1.0 | 0.4 |
| 8 | 0.7 | 0.5 |
| 9 | 0.8 | 0.4 |
| 10 | 0.7 | 0.4 |
| 11 | 0.6 | 0.2 |

### Example 2

Fatty Acid Oxidizing Enzymes

Four enzymes, viz. two laccases and two lipoxygenases were tested as described below. The laccase derived from Polyporus pinsitus had a MW by SDS-Page of 65 kDa, a pl by IEF of 3.5, and an optimum temperature at pH 5.5 of 60° C. The laccase derived from Coprinus cinereus had a MW by SDS-Page of 67-68 kDa, a pl by IEF of 3.5-3.8, and an optimum temperature at pH 7.5 of 65° C. The enzymes were prepared and purified as described in WO 96/00290 and U.S. Pat. No. 6,008,029. The two lipoxygenases were derived from Magnaporthie salvinii and Gaeumannomyces graminis, and they were prepared as described previously.

The enzyme dosage was adjusted to ensure maximum absorbancy increase per minute at 234 nm /530 nm, viz. in the range of 0.1-0.25 absorbancy units pr. min.

Substrate solution: 11.65 mg linoleic acid (60% Sigma), as well as 12.5 ml 0.56 mM Syringaldazine (Sigma) in ethanol was mixed with deionized water to a total volume of 25 ml.

50 microliter of the enzyme preparation to be tested was transferred to a quartz cuvette containing 900 microliters phosphate buffer (50 mM, pH 7.0) and 50 microliters of the substrate solution. The cuvette was placed in a spectrofotometer, thermostated at 23° C., and the absorbancies at 234 nm and 530 nm were measured as a function of time. The absorbancy at 530 nm is indicative of degradation of syringaldazine, whereas the absorbancy at 234 nm is indicative of degradation of linoleic acid. The absorbancy increase as a function of time is calculated on the basis of minutes 2 to 4 of the reaction time, i.e. d(A₂₃₄)/dt, as well as d(A₅₃₀)/dt.

The results are shown in Table 2 below. Of these four enzymes, only the two lipoxygenases qualify as a fatty acid oxidizing enzyme as defined herein. This is because RRD=Reaction Rate Difference=(dA₂₃₄/dt−dA₅₃₀/dt) is above zero only for these two enzymes.

TABLE 2

| | | | dA₂₃₄/dt − |
|---|---|---|---|
| | dA₅₃₀/dt | dA₂₃₄/dt | dA₅₃₀/dt |
| Enzyme | (units/min) | (units/min) | (units/min) |
| Polyporus pinsitus | 0.20 | 0.002* | −0.20 |
| laccase | | | |
| Magnaporthe salvinii | 0.0001* | 0.13 | 0.13 |
| lipoxygenase | | | |
| Coprinus cinereus | 0.17 | −0.001* | −0.17 |
| laccase | | | |
| Gaeumannomyces graminis | −0.03* | 0.21 | 0.21 |
| lipoxygenase | | | |

*this is equivalent to zero activity (analytical inaccuracy)

### Example 3

Acid Pretreatment of DDG

Prepare a 30% DS suspension of DDG and pretreat in a 1L Parr reactor. The suspension is made by weighing out 150 g dry weight of DDG, 7.5 g sulfuric acid (72 wt %) and 342.5 g of water. The final pH is between 1.3 and 1.5. The solids are then heated to 150° C. for 10 minutes. The resulting slurry is washed in a buchner funnel filtration system until the filtrate entering the vacuum flask reaches pH of 4.5. The acid pretreated DDG is referred to as “PDDG”.

### Example 4

Protease Treatment:

Prepare a 30% DS suspension of PDDG, pH 5.0. The suspension is prepared by weighing out 100 g dry weight of PDDG and mixing it with 50 mM sodium acetate, pH 5.0 until a final volume of 0.2 L is achieved. Add 0.1 g of ALCALASE™ 2.4L FG (0.27 AU/100 g dry weight of PDDG) with stirring. Mix the suspension at 250 rpm, 50° C. for 2 hours.

### Example 5

Fatty Acid Oxidizing Enzyme Treatment.

To the suspension prepared in Example 4, adjust the pH to 7.0 using 5.0 M ammonium hydroxide. Add 100 KU of Magnaporthe salvinii lipoxygenase (LOX) and mix the suspension at 250 rpm for 4 hours at 65° C.

### Example 6

Production of Ethanol from Enzyme-treated PDDG via an SSF Process

A mixture of 80 dry grams of liquefied corn mash and 20 dry grams PDDG is prepared and introduced into a 500 ml shake flask equipped with a trap for fermentation. The liquefied corn mash is prepared by a standard liquefaction procedure using a bacterial alpha-amylase A. The dextrose equivalent (DE) range of the liquefied mash is 10-12 DE. Water is used to adjust the final weight of the mash/PDDG slurry to 300 g. The pH of the slurry is adjusted to pH 5 with 40% (v/v) sulfuric acid. Ethanol Red yeast is propagated aerobically at 500 rpm and 32° C. for 8 hours. Glucoamylase SF (0.8 AGU/g DS) and yeast propagate (10 mL) are introduced into the slurry immediately before filling the shake flasks. The fermentation is carried out at 32° C. for 64 hours. The ethanol percentage is determined by HPLC and compared to the ethanol percentage from a corresponding control fermentation comprised of 80 dry grams of liquefied corn mash and 20 dry grams PDDG from Example 4 (protease-treated, no LOX treatment).

1-57. (canceled)

58. A process for producing ethanol, comprising the step of treating distillers' grains with a fatty acid oxidizing enzyme.

59. The process of claim 58, wherein the distillers' grains are further subject to a chemical treatment and/or a mechanical treatment.

60. The process of claim 58, wherein the distillers' grains are further subject to a chemical treatment and/or a mechanical treatment prior to the treatment with a fatty acid oxidizing enzyme.

61. The process of claim 58, wherein the chemical treatment comprises treating the distillers' grains with a mild acid.

62. The process of claim 58, wherein the mechanical treatment comprises treating the distillers' grains with a high temperature and a high pressure.

63. The process of claim 58, wherein starch is recovered from the treated distiller's grains.

64. The process of claim 63, wherein the starch recovered is treated with a starch degrading enzyme, esterase and/or hemicellulase or cellulase and fed into a liquefaction, sacoharfication and/or fermentation process.

65. The process of claim 58, wherein the starch is recovered and treated with a raw starch degrading enzyme and fed into a liquefaction, saccharfication and/or fermentation process.

66. The process of claim 58, wherein the recovered starch is treated with one or more enzymes selected from the group consisting of alpha-amylase, in particular acid alpha-amylases, CGTase, glucoamylase, maltogenic amylase, beta-amylase and fed into a liquefaction, saccharfication and/or fermentation process.

67. The process of claim 58, wherein the recovered starch is treated with a hemicellulase or cellulase and fed into a liquefaction, saccharfication and/or fermentation process.

68. The process of claim 58, wherein the recovered starch is treated with an esterase, preferably a lipolytic enzyme, such as a lipase or phospholipase and fed into a liquefaction, saccharfication or fermentation process.

69. The process of claim 58, wherein the recovered starch is treated with a glucoamylase and a fungal acid alpha-amylase and wherein said treated starch is fed into a liquefaction, saccharfication and/or fermentation process.

70. The process of claim 58, wherein the distillers grain are further treated with an enzyme selected from the group consisting of a maltogenic alpha-amylase, and an esterase, in particular a lipolytic enzyme, preferably a lipases or phospholipases.

71. The process of claim 58, wherein protein is recovered from the treated distiller's grains.

72. The process of claim 71, comprising treating the recovered protein with a protease and wherein said protease treated protein is fed into a liquefaction, saccharification and/or fermentation process.

73. The process of claim 71, wherein the protein is fed into a liquefaction process.

74. The process of claim 71, wherein the protease treated protein is fed into a simultaneous saccharification and fermentation process (SSF) or simultaneous liquefaction, saccharification and fermentation process.

75. The process of claim 58, wherein the distillers' grains are distillers' dried grains.

76. The process of claim 58, wherein the distliters' grains are distillers' dried grain with solubles.

77. The process of claim 58, wherein the distliters' grains are distliters' wet grains.
